(19) United States
(12) Patent Application Publication
(10) Pub. No.: US 2023/0087498 A1
(43) Pub. Date: Mar. 23, 2023

(54) A method for preparing a gaseous isotope reference, a method for determining an isotope ratio in a sample, and use of graphite for preparing a gaseous carbon and/or oxygen isotope reference

(71) Applicant: Teknologian tutkimuskeskus VTT Oy, Espoo (FI)

(72) Inventors: Teemu Kääriäinen, VTT (FI); Juho Salminen, VTT (FI); Albert Manninen, VTT (FI); Guillaume Genoud, VTT (FI)

(21) Appl. No.: 17/909,398

(22) PCT Filed: Mar. 3, 2021
(86) PCT No.: PCT/FI2021/050154

§ 371 Date: Sep. 5, 2022

(30) Foreign Application Priority Data
Mar. 3, 2020 (FI) 20205222

(51) Int. Cl. G01N 33/00; C01B 32/50; H01J 49/00

(52) U.S. Cl. CPC G01N 33/0011; C01B 32/50; H01J 49/0009; G01N 33/0006; C01P 2006/88; G01N 21/25

(57) ABSTRACT

According to an example aspect of the present invention, there is provided a method for preparing a gaseous isotope reference, the method comprising: providing a solid or liquid carbon-containing material exhibiting a carbon isotope ratio; providing oxygen gas or a gas mixture comprising oxygen gas, wherein said gas or gas mixture exhibits an oxygen isotope ratio; determining said carbon isotope ratio in the solid carbon-containing material and/or determining said oxygen isotope ratio in the oxygen gas or the gas mixture comprising oxygen; bringing the solid carbon-containing material in contact with the oxygen gas or the gas mixture comprising oxygen gas, in a high temperature in order to oxidize at least a part of the solid carbon-containing material to carbon dioxide to obtain the gaseous carbon and/or oxygen isotope reference in the form of carbon dioxide.

### FIELD

The present invention relates to isotopic reference materials that are used in isotopic analyses, and more particularly in isotopic analyses of stable isotopes or carbon and oxygen.

### BACKGROUND

Stable isotopes and their ratios are typically determined by using an isotope-ratio mass spectrometer (IRMS). Specific methods include static gas mass spectrometry, thermal ionization mass spectrometry (TIMS), secondary-ion mass spectrometry (SIMS) and multiple collector inductively coupled plasma mass spectrometry (MC-ICP-MS), and accelerator mass spectrometry (AMS).

The detected isotopic ratios are compared to a measured standard. For the stable carbon isotope ¹³C, the international standard (primary reference material) is prepared from a fossil belemnite found in the Peedee formation, referred to as VPDB (Vienna Pee Dee Belemnite), The ¹³C:¹²C ratio of VPDB is 0.0112372. The isotopic standard for oxygen is V-SMOW (Vienna Standard Mean Ocean Water).

Because the primary reference materials are not readily available, in-house standards (working standards) need to be prepared for calibration purposes. Such in-house materials are measured against the reference material, obtained in a small quantity. Such in-house working standards ultimately define the precision of isotope ratio measurements.

Thus, high precision isotope analysers are dependent on frequent calibrations with a sample exhibiting a known isotope ratio. The standards are typically in a gaseous form. However, this is not practical due to cumbersome logistics of gaseous materials. Large volumes of standard gases need to be transported to the analysis laboratory, particularly if several standards exhibiting different isotope ratios are desired.

There is a need for developing a new gaseous standard for isotope ratio measurements that will require smaller volumes and that can be generated on-site.

There is a further need for reducing the transportation costs of isotope standards.

There is a need for providing a more flexible and versatile method for preparing a series of working standards in order to analyse different samples involving a large variation in the isotope ratio to be determined. This is a particularly pertinent problem when using optical spectroscopy to determine isotopic compositions.

The embodiments of the present invention are intended to overcome at least some of the above discussed disadvantages and restrictions of the prior art.

### SUMMARY OF THE INVENTION

The invention is defined by the features of the independent claims. Some specific embodiments are defined in the dependent claims.

According to a first aspect of the present invention, there is provided a method for preparing a gaseous isotope reference, the method comprising: providing a solid or liquid carbon-containing material exhibiting a carbon isotope ratio; providing oxygen gas or a gas mixture comprising oxygen gas, wherein said gas or gas mixture exhibits an oxygen isotope ratio; bringing the solid carbon-containing material in contact with the oxygen gas or the gas mixture comprising oxygen gas, in a high temperature in order to oxidize at least a part of the solid carbon-containing material to carbon dioxide to obtain the gaseous carbon and/or oxygen isotope reference in the form of carbon dioxide.

Various embodiments of the first aspect may comprise at least one feature from the following bulleted list:
The method further comprises, before said oxidation step, determining a carbon isotope ratio in the solid carbon-containing material and/or determining an oxygen isotope ratio in the oxygen gas or the gas mixture comprising oxygen.
Said gaseous isotope reference is capable of acting as a reference in an isotope ratio analysis in which an isotope ratio in a sample is determined.
The solid carbon-containing material is graphite.
The carbon isotope ratio is the ratio of ¹³C to ¹²C.
The oxygen isotope ratio is the ratio of ¹⁸O to ¹⁶O or the ratio of ¹⁷O to ¹⁶O.
The oxidation step is conducted in a temperature of 500 to 800° C., for example at least 550° C.
The solid carbon-containing material is brought in contact with a gas mixture comprising at least 1% O₂.
The amount of carbon monoxide generated in the oxidation step is less than 5%, preferably less than 0.5%.
The determining step is carried out by using optical spectroscopy, such as laser spectroscopy, or by mass spectrometry.
At least two gaseous isotope references are prepared, each of said references exhibiting a different isotopic ratio; the method comprising:
   providing a first solid carbon-containing material exhibiting a first ratio of ¹³C to ¹²C;
   optionally, determining the ratio of ¹³C to ¹²C in the first solid carbon-containing material;
   providing a second solid carbon-containing material exhibiting a second ratio of ¹³C to ¹²C, whereby the second ratio is different from the first ratio;
   optionally, determining the ratio of ¹³C to ¹²C in the second solid carbon-containing material;
   bringing the first solid carbon-containing material in contact with oxygen gas or a gas mixture comprising oxygen gas, in a high temperature in order to oxidize at least a part of the first solid carbon-containing material to carbon dioxide, to prepare a first gaseous carbon isotope reference;
   bringing the second solid carbon-containing material in contact with oxygen gas or a gas mixture comprising oxygen gas, in a high temperature in order to oxidize at least a part of the second solid carbon-containing material to carbon dioxide, to prepare a second gaseous carbon isotope reference.
The obtained gaseous carbon and/or oxygen isotope reference exhibits a determined carbon isotope ratio and/or a determined oxygen isotope ratio.
The obtained gaseous carbon and/or oxygen isotope reference provides a stable isotope ratio, preferably to account for drift errors in an optical isotope measurement instrumentation that is used in an isotope ratio analysis of a sample.

According to a second aspect of the present invention, there is provided a method for determining a stable isotope ratio in a sample, the method comprising: preparing a gaseous isotope reference exhibiting a determined carbon or oxygen isotope ratio by the method according to the first aspect; providing a gaseous sample exhibiting a carbon isotope ratio and/or an oxygen isotope ratio to be determined; carrying out a carbon isotope ratio analysis and/or an oxygen isotope ratio analysis on the gaseous sample, in which analysis said gaseous isotope reference is used as a reference.

Various embodiments of the second aspect may comprise at least one feature from the following bulleted list:
The isotope ratio analysis or analyses are carried out by using optical spectroscopy, such as laser spectroscopy, or by mass spectrometry.
The gaseous sample originates from human breath.
The isotope ratio analysis is an analysis of the ratio of ¹³C to ¹²C, or an analysis of the ratio of ¹⁸O to ¹⁶O, or an analysis of the ratio of ¹⁷O to ¹⁶O in the gaseous sample, or any combination thereof.

According to a third aspect of the present invention, there is provided use of graphite for preparing a gaseous carbon and/or oxygen isotope reference.

Various embodiments of the third aspect may comprise at least one feature from the following bulleted list:
Graphite is used for preparing several carbon isotope references, each of the references exhibiting a different carbon isotope ratio ¹³C to ¹²C.
Said isotope reference is in the form of carbon dioxide.

### ADVANTAGES OF THE INVENTION

At least some embodiments of the present invention provide a compact instrument for on-site and field measurements.

The present invention makes it possible to reduce the cost and logistical burden of calibration standards for optical isotope analysers. Also, the physical size of standard containers can be reduced compared to standard containers containing pressurized gas.

The present invention provides an improved gaseous standard for isotope ratio analyses. The present invention reduces the transport and storage costs of in-house working standards suitable for optical spectroscopic methods.

At least some embodiments of the present invention enable the preparation of both carbon and oxygen isotope standards on-site and by using the same apparatus.

At least some embodiments of the present invention enable the preparation of several carbon isotope standards exhibiting different carbon isotope ratios.

A significant advantage of the present method is that it can provide compact instrumentation and compact gas standards. It is also possible to retain the advantages provided by the use of optical methods.

The present invention provides advantages in connection with any isotope analysis method, such as optical methods or mass spectrometric methods.

### BRIEF DESCRIPTION OF THE DRAWINGS

FIG. 1 illustrates the preparation of a gas standard in accordance with at least some embodiments of the present invention;

FIG. 2 shows the results from isotope analyses for gaseous products prepared in accordance with at least some embodiments of the present invention.

FIG. 3 shows the amount of carbon dioxide formed in accordance with at least some embodiments of the present invention.

### EMBODIMENTS

In the present context, the term “isotope standard” and “isotope reference” are used synonymously and refer to a material exhibiting a substantially constant and/or repeatable isotope ratio. The terms “standard”, “working standard”, “reference” and “working reference” can be used interchangeably in the present context.

In the following, all % values refer to vol-% values unless otherwise stated.

In the present invention, the required gas standard (working standard) is generated by using graphite as the starting material. Graphite, as a solid material, is easy to transport to the laboratory that is in need of calibration of a spectrometer for isotope ratio measurements. The gas standard is prepared from graphite by converting it to a gaseous form, for example by oxidizing it to carbon dioxide. The gaseous end product exhibits the same isotope ratios as the carbon and optionally the oxygen participating in the conversion reaction.

In some embodiments, the oxygen-containing carrier gas that is used in the oxidation reaction can be drawn from ambient or laboratory air or from a cylinder containing normal pressurised air. The graphite is heated and a constant flow of carrier gas is brought in contact with the graphite. The oxygen in the carrier gas and the carbon in the graphite generate both CO₂ and CO. The constant isotope ratios ¹³C:¹²C and ¹⁸O:¹⁶O in the graphite and in the air will result in constant and repeatable isotope ratios in the generated carbon dioxide and carbon monoxide molecules.

In preferred embodiments, the solid carbon-containing starting material is graphite. In other embodiments, another carbon allotrope may be used. In some embodiments, any solid or liquid (non-gaseous) carbon-containing material or compound that is capable of being oxidized to carbon dioxide can serve as the solid starting material in a method according to the present invention. Preferably the oxidation reaction produces carbon dioxide as the only carbon-containing product of the oxidation reaction. Preferably the carbon isotope ratio in the produced carbon dioxide is the same or substantially the same as in the solid carbon-containing starting material.

In some embodiments, the produced oxidized and gaseous carbon-containing material comprises or consists of carbon dioxide and/or carbon monoxide.

Typically, graphite samples from different sources will exhibit a different isotopic signature. This fact can be utilized in the present invention in order to prepare a series of stable carbon isotope standards exhibiting different isotope ratios ¹³C:¹²C. In such embodiments, the gaseous isotope standards are prepared by selecting graphite samples exhibiting different isotope ratios to serve as the starting materials. Preferably, the isotope ratio range in the generated standards overlaps the isotope ratios in the actual samples to be determined to provide high accuracy.

In some embodiments, at least two gaseous isotope standards are prepared, each of said standards comprising a different isotopic ratio. The method comprises providing a first solid carbon-containing material exhibiting a first ratio of ¹³C to ¹²C; determining the ratio of ¹³C to ¹²C in the first solid carbon-containing material; providing a second solid carbon-containing material exhibiting a second ratio of ¹³C to ¹²C, whereby the second ratio is different from the first ratio; determining the ratio of ¹³C to ¹²C in the second solid carbon-containing material; bringing the first solid carbon-containing material in contact with oxygen gas or a gas mixture comprising oxygen gas, in a high temperature in order to oxidize at least a part of the first solid carbon-containing material to carbon dioxide, to prepare a first gaseous carbon isotope standard; bringing the second solid carbon-containing material in contact with oxygen gas or a gas mixture comprising oxygen gas, in a high temperature in order to oxidize at least a part of the second solid carbon-containing material to carbon dioxide, to prepare a second gaseous carbon isotope standard.

Here, the first and second gaseous isotope standards are prepared separately, by utilizing the first and second carbon-containing starting materials with different carbon stable isotope signatures.

In preferred embodiments, the solid carbon-containing starting material is oxidized to generate a gaseous carbon-containing material. The oxidant is preferably oxygen comprised in an oxygen-containing gas mixture or in the form of pure oxygen gas. Alternative, other oxygen-containing oxidants may be used, such as ozone.

In embodiments employing oxygen as the oxidant, the solid carbon-containing material is preferably heated to a high temperature. Preferably, the temperature is at least 450° C., more preferably at least 500° C., even more preferably at least 550° C., such as 550 to 700° C. or 500 to 650° C.

In temperatures below 900° C., the amount of CO generated stays low.

In temperatures 550 to 650° C., the isotope ratios ¹³C:¹²C and/or 18O:¹⁶O in the produced gas are constant and repeatable and the Allan variance is good.

In one embodiment, graphite is heated to high temperature, preferably to at least 500° C. A constant and repeatable steady flow of oxygen-containing carrier gas is flown through the system containing the heated graphite. A gas matrix containing both CO₂ and CO is generated. The CO₂/CO ratio can be adjusted by altering the oxidation temperature. The isotope ratio of the generated gas is measured by isotope-selective optical spectroscopy. Then, the generated CO₂/CO gas mixture can be used for the calibration of an optical isotope analyser.

For example, by using the present invention and 1 gram of graphite as the starting material, it is possible to replace about 2 litres of 100% pure conventional CO₂ reference gas.

In some embodiments, the generated gaseous isotope standard is a stable carbon isotope standard for ¹³C:¹²C isotope ratio analyses. Any carrier gas comprising an oxidant can be used as long as it is capable of oxidizing graphite to carbon dioxide. Suitable carrier gases comprising an oxidant include ambient air, synthetic air, pressurized normal air, gas mixtures comprising oxygen, and pure oxygen. Preferably the carrier gas does not contain substantial amounts of such carbon sources that would have a different carbon isotope ratio from the ratio in the starting material, preferably graphite. Most preferably, the carrier gas does not contain carbon dioxide. Undesired carbon dioxide can be removed from the carrier gas, such as room air, by filtering.

In some embodiments, the generated gaseous isotope standard is a stable carbon isotope standard for ¹³C:¹²C isotope ratio analyses. Any carrier gas comprising a reactant can be used as long as it is capable of converting graphite or some other solid carbon-containing compound to a sole gaseous carbon-containing compound so that no other carbon-containing compounds are generated to any significant extent, for example as amounts exceeding 2%.

In some embodiments, the carrier gas or gas mixture comprises a reactant that is capable of reacting with a non-gaseous carbon-containing compound to produce a gaseous carbon-containing compound.

Preferably the carrier gas does not contain any significant amounts of carbonaceous compounds, or at least any such carbon sources that would have a different carbon isotope ratio from the ratio in the solid carbon-containing starting material, such as graphite.

In some embodiments, the generated gaseous isotope standard is a stable isotope standard for both ¹³C:¹²C and ¹⁸O:¹⁶O isotope ratio analyses, preferably it is carbon dioxide or carbon monoxide exhibiting known isotope ratios ¹³C:¹²C and ¹⁸O:¹⁶O. Preferred carrier gases include gas mixtures comprising oxygen, such as a gas mixture consisting of O₂ and N₂, or pure oxygen. Most preferably, the carrier gas is an oxygen-containing gas mixture exhibiting a known isotope ratio ¹⁸O:¹⁶O or ¹⁷O:¹⁶O. An advantage of this embodiment is that the same method and the same apparatus can be utilized for preparing both isotope standards (carbon and oxygen), either in separate steps or simultaneously.

In some embodiments, the generated gaseous isotope standard is a stable oxygen isotope standard for ¹⁸O:¹⁶O or ¹⁷O:¹⁶O isotope ratio analyses. Here the carrier gas comprising an oxidant shall contain a stable isotope ratio of oxygen. Suitable carrier gases comprising an oxidant include gas mixtures comprising oxygen, such as synthetic gas mixtures comprising oxygen, and pure oxygen.

In some embodiments, the solid carbon-containing material is brought in contact with a gas mixture comprising at least 1% O₂, preferably at least 2% O₂, for example 2% to 50% O₂.

In one embodiment, the gas mixture comprises oxygen and nitrogen.

In some embodiments, the isotope analysis method is an optical method, such as a laser spectroscopic method.

In some other embodiments, the isotope analysis method is a mass spectroscopic method.

The present invention can be applied for example for emission monitoring, atmospheric sensing and breath analysis.

An example application of the present invention is long-term (hours to days) monitoring of exhaled breath isotopologues, for example breath isotope analysis for mechanically ventilated patients for sepsis detection.

### EXAMPLE 1

In one embodiment, the system according to the present invention comprises a graphite reference gas generator, where CO₂ is produced either statically or with a continuous flow of O₂ containing carrier gas. The generated CO₂ has stable isotopic ratios.

The reference gas generator is connected to a laser based isotope analyser.

The isotope analyser will compare the known reference (generated from graphite) to an unknown CO₂ sample. Based on the difference in readout signal, delta is calculated for the unknown sample. The laser spectrometer reading may drift, but it will drift the same amount for the reference and for the CO₂ sample, so the difference is always a valid reading.

The above method is highly advantageous in comparison to known methods in which the reference gas generator is in the form of a pressurised gas cylinder.

### EXAMPLE 2

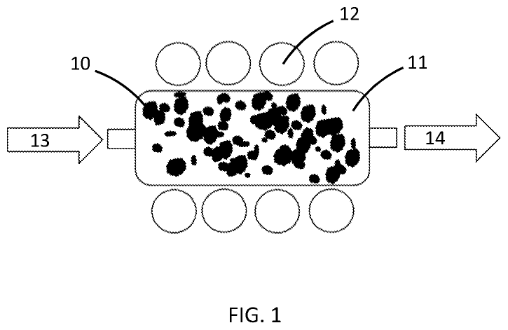

The stability of isotope fractionation in the method according to an embodiment of the present invention was tested. The results are shown in FIGS. 1 to 3.

FIG. 1 illustrates the preparation of a gas standard. Pure graphite 10 was placed inside a chamber 11. Outside the chamber, there are heating rods 12 that were used for heating the chamber and thus the graphite. An oxygen-containing carrier gas 13 with a known composition was flown through the chamber in order to oxidize the graphite. The product was a gas mixture 14 comprising both CO₂ and CO. The CO₂ and CO molecules exhibited stable isotopic ratios ¹³C/¹²C and ¹⁸O/¹⁶O.

In this example, the graphite was heated to 550° C. Carrier gas (2% O₂ and 98% N₂) was flown through the chamber. The carbon in the graphite was oxidized mainly to CO₂.

The generated gas mixture can be used as a standard in a calibration of an isotope analyser. The isotope analysis method may be an optical method, such as a laser spectroscopic method, or a mass spectroscopic method.

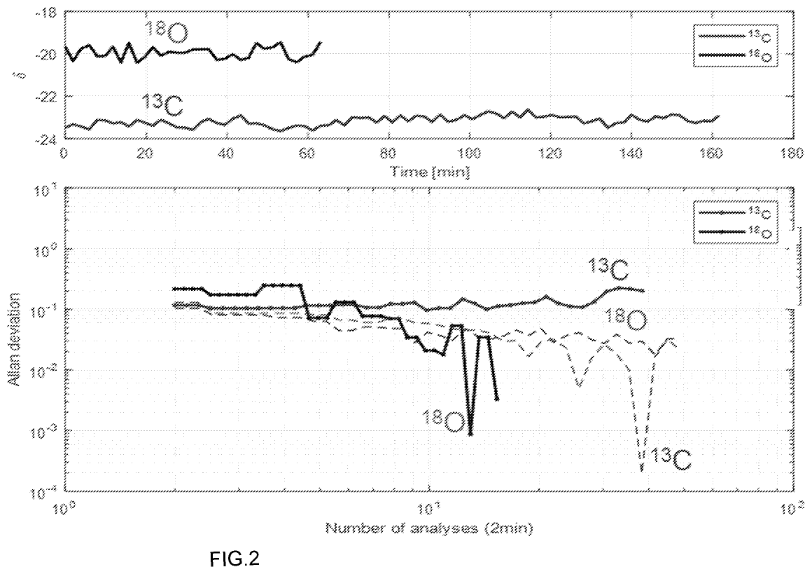

The isotopic ratios ¹³C:¹²C and ¹⁸O:¹⁶O present in the produced gas were monitored. The product, a gas mixture containing mainly CO₂, was analysed by a CO₂ stable isotope analyser (VTT). The stability of the resulted isotope ratios was determined via Allan deviation analysis. FIG. 2 shows the results from these isotope analyses.

In FIG. 2, the upper graph is the time series of the isotope ratio (δ) measurements. The lower graph is an Allan deviation plot. In the lower graph, the solid lines are the results for the gas prepared from the graphite. The dashed lines are the results for reference measurements in which a normal technical air gaseous standard was used.

In the upper graph, the lower line depicts the isotope ratio ¹³C:¹²C as a function of time, and the upper line depicts the isotope ratio ¹⁸O:¹⁶O as a function of time.

In the lower graph, the solid lines depict the Allan deviation for the ¹³C:¹²C analysis and for the ¹⁸O:¹⁶O analysis.

It was observed that for the isotope ratio ¹⁸O:¹⁶O in CO₂, the precision was well within the measurement accuracy. The isotope ratio ¹³C:¹²C in CO₂ was less stable, but still within 0.2‰.

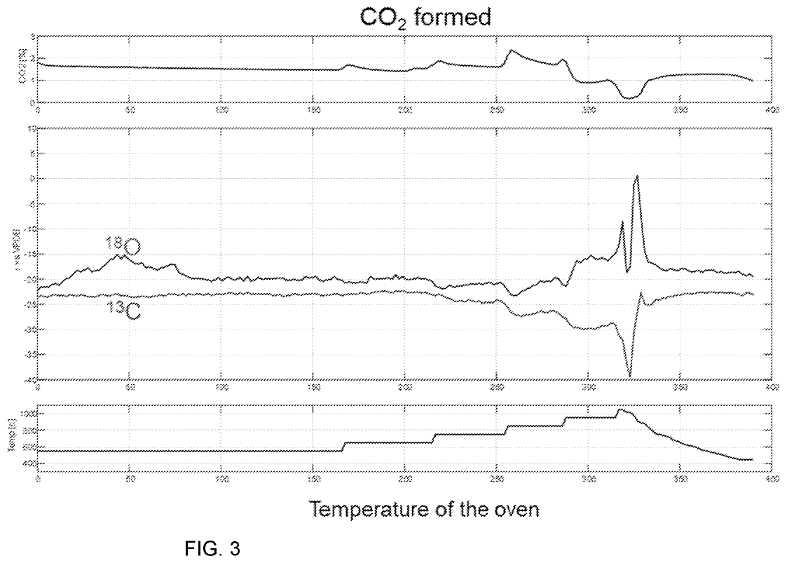

In FIG. 3, the topmost graph shows the amount of carbon dioxide formed during the heating: CO₂ (%) as a function of time in minutes. In FIG. 3, the middle graph shows the isotopic ratios ¹³C:¹²C and ¹⁸O:¹⁶O: δ vs VPDB as a function of time in minutes. In FIG. 3, the lowermost graph shows the temperature of the oven (° C.) as a functional of time in minutes.

FIG. 3 shows that CO₂ production decreased when going to temperatures above 800° C., and a more pronounced decrease could be observed in temperatures above 900° C. It was observed that it is advantageous to use a temperature in the range 500 to 800° C., and preferably a temperature of at least 550° C.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### INDUSTRIAL APPLICABILITY

The present invention is industrially applicable at least for the preparation of gaseous isotope standards for isotope ratio analyses.

### ACRONYMS LIST

| | |
|---|---|
| IRMS | isotope-ratio mass spectrometer |
| TIMS | thermal ionization mass spectrometry |
| SIMS | secondary-ion mass spectrometry |
| MC-ICP-MS | multiple collector inductively coupled |
| | plasma mass spectrometry |
| AMS | accelerator mass spectrometry |
| VPDB | Vienna Pee Dee Belemnite |
| V-SMOW | Vienna Standard Mean Ocean Water |

### REFERENCE SIGNS LIST

10 graphite
11 chamber
12 heating rod
13 carrier gas
14 product gas mixture

1-20. (canceled)

21. A method for preparing a gaseous carbon and/or oxygen isotope reference, the method comprising:
providing a solid or liquid carbon-containing material exhibiting a carbon isotope ratio;
providing oxygen gas or a gas mixture comprising oxygen gas, wherein said gas or gas mixture exhibits an oxygen isotope ratio;
bringing the solid or liquid carbon-containing material in contact with the oxygen gas or the gas mixture comprising oxygen gas at elevated temperature to oxidize at least a part of the solid or liquid carbon-containing material to carbon dioxide to obtain the gaseous carbon and/or oxygen isotope reference in the form of carbon dioxide.

22. The method according to claim 21, further comprising, before said oxidation step, determining said carbon isotope ratio in the solid or liquid carbon-containing material and/or determining said oxygen isotope ratio in the oxygen gas or the gas mixture comprising oxygen; and wherein said gaseous carbon and/or oxygen isotope reference is capable of acting as a reference in an isotope ratio analysis in which an isotope ratio in a sample is determined.

23. The method according to claim 21, wherein the solid or liquid carbon-containing material is graphite.

24. The method according to claim 21, wherein the carbon isotope ratio is a ratio of ¹³C to ¹²C.

25. The method according to claim 21, wherein the oxygen isotope ratio is a ratio of ¹⁸O to ¹⁶O or a ratio of ¹⁷O to ¹⁶O.

26. The method according to claim 21, wherein the oxidation step is conducted in a temperature of 500 to 800° C.

27. The method according to claim 21, wherein the solid or liquid carbon-containing material is brought in contact with a gas mixture comprising at least 1% O₂.

28. The method according to claim 21, wherein the amount of carbon monoxide generated in the oxidation step is less than 5%.

29. The method according to claim 21, wherein the determining step is carried out by using optical spectroscopy or by mass spectrometry.

30. The method according to claim 21, wherein at least two gaseous isotope references are prepared, each of said at least two gaseous isotope references exhibiting a different isotopic ratio; the method comprising:
providing a first solid carbon-containing material exhibiting a first ratio of ¹³C to ¹²C;
providing a second solid carbon-containing material exhibiting a second ratio of ¹³C to ¹²C, wherein the second ratio is different from the first ratio;
bringing the first solid carbon-containing material in contact with oxygen gas or a gas mixture comprising oxygen gas at elevated temperature to oxidize at least a part of the first solid carbon-containing material to carbon dioxide, to obtain a first gaseous carbon isotope reference in the form of carbon dioxide;
bringing the second solid carbon-containing material in contact with oxygen gas or a gas mixture comprising oxygen gas at elevated temperature to oxidize at least a part of the second solid carbon-containing material to carbon dioxide, to obtain a second gaseous carbon isotope reference in the form of carbon dioxide.

31. The method according to claim 21, wherein the obtained gaseous carbon and/or oxygen isotope reference exhibits a determined carbon isotope ratio and/or a determined oxygen isotope ratio.

32. The method according to claim 21, wherein the obtained gaseous carbon and/or oxygen isotope reference provides a stable isotope ratio to account for drift errors in an optical isotope measurement instrumentation used in an isotope ratio analysis of a sample.

33. A method for determining a stable isotope ratio in a sample, the method comprising:
preparing a gaseous carbon and/or oxygen isotope reference exhibiting a determined carbon and/or oxygen isotope ratio by the method according to claim 1;
providing a gaseous sample exhibiting a carbon isotope ratio and/or an oxygen isotope ratio to be determined; and
carrying out a carbon isotope ratio analysis and/or an oxygen isotope ratio analysis on the gaseous sample, in which said carbon isotope and/or oxygen isotope ratio analysis, said gaseous carbon and/or oxygen isotope reference is used as a reference.

34. The method according to claim 33, wherein the isotope ratio analysis is carried out by using optical spectroscopy or by mass spectrometry.

35. The method according to claim 33, wherein the gaseous sample originates from atmosphere or human breath.

36. The method according to claim 33, wherein the carbon isotope and/or oxygen isotope ratio isotope ratio analysis is an analysis of a ratio of ¹³C to ¹²C, or an analysis of a ratio of ¹⁸O to ¹⁶O, or an analysis of a ratio of ¹⁷O to ¹⁶O in the gaseous sample, or any combination thereof.

37. The method according to claim 36, wherein the isotope ratio analysis is an analysis of the ratio of ¹⁸O to ¹⁶O, or an analysis of the ratio of ¹⁷O to ¹⁶O in the gaseous sample, or any combination thereof.

38. The method according to claim 33, wherein the gaseous carbon and/or oxygen isotope reference is formed from the oxidation of graphite.
